(19) United States
(12) Patent Application Publication
(10) Pub. No.: US 2026/0004897 A1
(43) Pub. Date: Jan. 1, 2026

(54) SYSTEM AND METHOD FOR DESIGNING QUALITY CONTROL (QC) RANGES FOR MULTIPLE CLINICAL DIAGNOSTIC INSTRUMENTS TESTING THE SAME ANALYTE

(71) Applicant: Bio-Rad Laboratories, Inc., Hercules, CA (US)

(72) Inventors: John C. Yundt-Pacheco, Fairview, TX (US); Curtis A. Parvin, Garland, TX (US)

(73) Assignee: Bio-Rad Laboratories, Inc., Hercules, CA (US)

(21) Appl. No.: 18/992,139

(22) PCT Filed: Jul. 18, 2023
(86) PCT No.: PCT/US23/70419

§ 371 Date: Jan. 7, 2025

Related U.S. Application Data
(60) Provisional application No. 63/368,994, filed on Jul. 21, 2022.

(51) Int. Cl. G16H 10/40; G01N 35/00

(52) U.S. Cl. CPC G16H 10/40; G01N 35/00623; G01N 35/00871; G01N 2035/009; G01N 2035/0091

(57) ABSTRACT

Systems and methods for performing testing of a single analyte on a group of multiple clinical diagnostic analyzers, or a single clinical diagnostic analyzer having multiple analytic units, or combinations thereof, are disclosed. A mean and SD for each individual instrument are input to at least one of the instruments along with a QC rule to be used, the probability of false rejection function for the QC rule, and a desired false rejection rate. A group mean and a group SD are calculated to satisfy the desired false rejection rate and QC rule and loaded into each individual instrument for use in testing the single analyte at each individual instrument.

### CROSS-REFERENCE TO RELATED APPLICATIONS

This application is a 371 national phase of, and claims the benefit of, International Application Number PCT/US23/70419, filed Jul. 18, 2023, and U.S. Provisional Application Ser. No. 63/368,994, filed Jul. 21, 2022, the disclosure of which is hereby incorporated herein in its entirety by reference.

### BACKGROUND OF THE INVENTION

The present invention relates to generally to clinical diagnostic processes, and more particularly to systems and methods for designing and implementing quality control (QC) ranges for multiple clinical diagnostic analyzers and systems testing the same analyte.

In real-world laboratory testing, it is common for clinical diagnostic laboratories to use multiple instruments or analyzers to test the same analyte or test specimen. In some cases, this results from the volume of patient specimens to be tested—i.e., there are too many patient specimens to be tested on a single diagnostic analyzer instrument in an acceptable time frame. In other cases, the diagnostic analyzer instrument itself may comprise multiple analytic units testing the same analyte—i.e., the analyzer itself is essentially comprised of multiple analyzers within the same unit's housing or cabinet.

Known QC design methodology focuses primarily on QC procedures for analytes being tested on a single instrument in the laboratory, with the QC design usually requiring typically requiring implementation of: a QC target for the instrument (typically the mean of the QC evaluations); a QC range for the instrument (typically the standard deviation (SD) of the QC evaluations); and a QC rule to implement on the instrument (typically as a function of the QC target and QC range) which determines if a QC result should be accepted or rejected.

For example, a representative QC design for a single instrument may include: a QC target=100 (from the mean of the response of the QC material on the instrument); a QC range=15 (from the SD of the responses of the QC material on the instrument); and a 1:2s QC rule (where a QC results is accepted if it is within +/−2 QC range of the QC target.

Traditional QC design provides no or little guidance for designing a QC method for an analyte that is tested on multiple instruments (or tested on multiple analytic units within the same analyzer). Thus, faced with testing a single analyte on multiple instruments, most laboratories and clinicians simply test the analyte on several different individual instruments and attempt to aggregate the results of those separate tests after the testing is completed. However, such aggregation inevitably leads to unsatisfactory results as there is no accounting for differences between the instruments themselves, only an attempt to compensate for varying results after the fact. Thus, a higher and/or lower reading by one or two of the instruments can skew the mean such that the number of false rejections is increased over what would have been expected if the analyte had been tested on a single instrument.

Thus, it can be seen that there remains a need in the art for effective QC systems and methods method that address the issue of testing an analyte on multiple instruments.

### BRIEF SUMMARY OF THE INVENTION

The present invention is directed to systems and methods for designing and implementing quality control (QC) ranges for multiple clinical diagnostic instruments testing the same analyte.

While numerous QC design systems and methods are known to ensure the accuracy of clinical diagnostic processes and analyzers used to test analytes, those known QC schemes address the testing of an individual analyte on an individual QC instrument, such as a clinical diagnostic analyzer. However, many real-world scenarios dictate that an analyte may, in fact, be tested on multiple analyzers, or on an analyzer having multiple analytic units. The use of multiple instruments raises the issue of the selection of suitable parameters to be used or applied across a collection of multiple instruments, such as suitable standard deviation (SD) or suitable mean to apply across the collection of multiple instruments. For example, it is not known in the prior art to design a QC range where the false rejection rate of the collection of instruments testing the same analyte using a QC target of the mean of the instruments matches the false rejection rate of the collection of instruments where the QC targets are set to the individual instrument means, and the QC ranges are set to the individual instrument SD's.

Embodiments of the systems and methods of the present invention as described herein addresses the SD and mean selection problem and further describe how to design and implement QC ranges for multiple instruments testing the same analyte, wherein the QC target is the mean of the group of instruments, and the QC range is a single value selected so that for a given QC rule, the average false rejection rate for the collection of instruments matches a desired design parameter.

In an exemplary embodiment, a group of multiple clinical diagnostic analyzers, or a single clinical diagnostic analyzer having multiple analytic units, or combinations thereof, are employed to test a single analyte—i.e., a single analyte is tested on multiple clinical diagnostic analyzers. The mean and SD for each analyzer/instrument (and/or each analytic unit) are input to at least one of the instruments along with a QC rule to be used, the probability of false rejection function for the QC rule, and a desired false rejection rate and a group mean and a group SD are calculated that satisfy the desired false rejection rate and QC rule.

In one aspect, QC ranges for each of the individual instruments are set from a single group SD so that the false rejection rate is allocated to each instrument proportionately to their performance (a false rejection is when nothing is wrong with the test method, but the QC result is rejected). Thus, the worst performing instrument (i.e., the instrument with the highest SD) will have the largest allocation of false rejections, which is highly desirable as lessening the influence of the worst performing instrument likewise reduces the worst-case risk of patient harm from erroneous results as compared to using individual instrument QC means. By contrast, when QC ranges are set from individual instrument SDs, as is currently the case in the prior art, the false rejection rate of each instrument will be uniformly distributed.

In one aspect, a QC range for the collection of instruments testing the same analyte is determined such that the average false rejection rate of the collection of instruments meets the desired false rejection rate criteria.

In another aspect, a group mean and a group standard deviation are calculated from the means and standard deviations of the group of individual instruments (and/or analytic units) being used to test the analyte. In one aspect, the calculated group mean and group standard deviation are then used as the mean and SD on each individual instrument for testing the single analyte.

In a further aspect, the probability of false rejection is determined for a given QC rule. In one aspect, the probability of false rejection is calculated from the QC target, QC range, and the individual mean and standard deviation of each of the instruments in the group.

In another aspect, the calculated group mean and group SD is provided to each clinical diagnostic analyzer in the group. In a further aspect, each clinical diagnostic analyzer or instrument is a specialized device for testing analytes, and may include a processor, memory, measurement hardware, and an input panel/display. Each analyzer may prompt a user to begin a test of the analyte and prompts a user to load, or automatically loads, the analyte and instigates testing and analysis of the analyte.

In another aspect, upon completion of the test of the analyte, each instrument in the group stores the results and/or provides the results to a central server for further presentation and/or analysis.

In another aspect, a group of laboratories, each comprising one or more clinical diagnostic analyzers, are in communication with each other in a peer group configuration, where information and data is shared between the members of the peer group. Shared information may comprise data regarding analyses performed and information relating to analyte testing data and/or quality control material used in the laboratory.

In another aspect, the use of a group mean and a group SD provide an improvement to the technological fields of clinical diagnostic testing and analyte testing fields and allow a laboratory to minimize the likelihood of reporting erroneous patient results as compared to using individual instrument mean and SD values. Minimized numbers of erroneous patient results reduces retesting and saves time, labor, and material costs as compared to systems and methods known in the prior art.

Reference to the remaining portions of the specification, including the drawings and claims, will realize other features and advantages of the present invention. Further features and advantages of the present invention, as well as the structure and operation of various embodiments of the present invention, are described in detail below with respect to the accompanying drawings and claims. In the drawings, like reference numbers indicate identical or functionally similar elements.

### BRIEF DESCRIPTION OF THE DRAWINGS

The present invention will be described in greater detail in the following detailed description of the invention with reference to the accompanying drawings that form a part hereof, in which:

FIG. 1 depicts a block diagram of a clinical diagnostic analyzer system having a plurality of clinical diagnostic analyzers in communication with a server over a network in accordance with an exemplary embodiment of the present invention.

FIG. 2 depicts a block diagram of a single clinical diagnostic analyzer of the system of FIG. 1.

FIG. 3A is a depiction of a first exemplary prompt screen presented by the clinical diagnostic analyzer of FIG. 2.

FIG. 3B is a depiction of a second exemplary prompt screen presented by the clinical diagnostic analyzer of FIG. 2.

FIG. 3C is a depiction of a third exemplary prompt screen presented by the clinical diagnostic analyzer of FIG. 2.

FIG. 3D is a depiction of a fourth exemplary prompt screen presented by the clinical diagnostic analyzer of FIG. 2.

FIG. 4 is a block diagram of a plurality of clinical diagnostic analyzers as in FIG. 1 arranged in a peer group configuration.

FIG. 5 is a flow diagram of an exemplary method for conducting testing of a single analyte on a plurality of clinical diagnostic instruments in accordance with an exemplary embodiment of the present invention.

### DETAILED DESCRIPTION OF EXEMPLARY EMBODIMENTS

Systems and methods for designing quality control (QC) ranges for multiple clinical diagnostic analyzer instruments testing the same analyte in accordance with exemplary embodiments of the present invention are described herein. While the invention will be described in detail hereinbelow with reference to the depicted exemplary embodiments and alternative embodiments, it should be understood that the invention is not limited to the specific configurations shown and described in these embodiments. Rather, one skilled in the art will appreciate that a variety of configurations may be implemented in accordance with the present invention. As used herein, the terms “clinical diagnostic analyzer”, “analyzer”, “instrument”, and variations thereof may be used to refer to the specialized diagnostic device for testing analytes, patient specimens, and the like to determine various characteristics of the analyte.

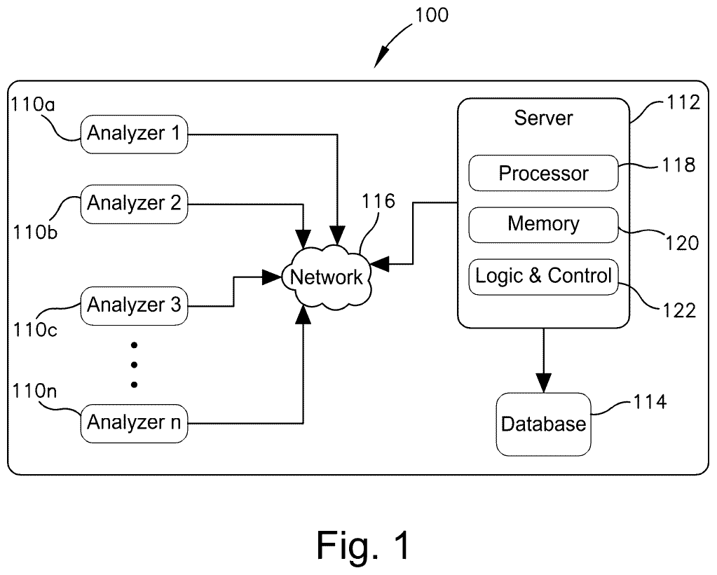

Looking first to FIG. 1, a clinical diagnostic system in accordance with an exemplary embodiment of the present invention is depicted generally by the numeral 100. The system 100 generally includes a plurality of clinical diagnostic analyzers 110a, 110b, 110c, 110n and a server 112 in communication with a database 114. The group of clinical diagnostic analyzers 110a, 110b, 110c, 110n are in communication with network 116, which facilitates the transmission of instructions, information, and data between each clinical diagnostic analyzer 110a, 110b, 110c, 110n and the server 112, as well as between each of the clinical diagnostic analyzers 110a, 110b, 110c, 110n and any of the other diagnostic analyzers, or between any combination of clinical diagnostic analyzers and/or the server. It should be understood that the number of analyzers may be any number from 1 to n, such as groups of 2, 6, 10, or 100 analyzers.

Network 116 may be any local area network (LAN), wide area network (WAN), ad-hoc network, or other network configuration known in the art, or combinations thereof. For example, in the exemplary embodiment depicted in FIG. 1, network 116 may include a LAN allowing communication between the clinical diagnostic analyzers 110a, 110b, 110c, 110n, such as in a single laboratory setting having multiple clinical diagnostic analyzers, an may also include a WAN, such as the Internet or other wide area network, allowing communication between the LAN and the server 112 and/or between the clinical diagnostic analyzers and the server.

It should be understood that the configurations depicted in FIG. 1 is exemplary, and not limiting, and that the invention as described herein may be embodied in a single clinical diagnostic analyzer, in a group of clinical diagnostic analyzers co-located in a single laboratory or facility, and in group of clinical diagnostic analyzers that are geographically dispersed to allow a group of thus interconnected clinical diagnostic analyzers to each test a single analyte.

For example, multiple systems 100, each comprising one or more clinical diagnostic analyzers and servers may be located in a single laboratory, or in multiple laboratories dispersed across a facility or across the globe, all in communication via a WAN. It should be further understood that the present invention may be embodied in a single clinical diagnostic analyzer, or in a group of clinical diagnostic analyzers in communication with each other over a LAN or WAN, without a server or servers. These and other variations and embodiments will be apparent to those skilled in the art.

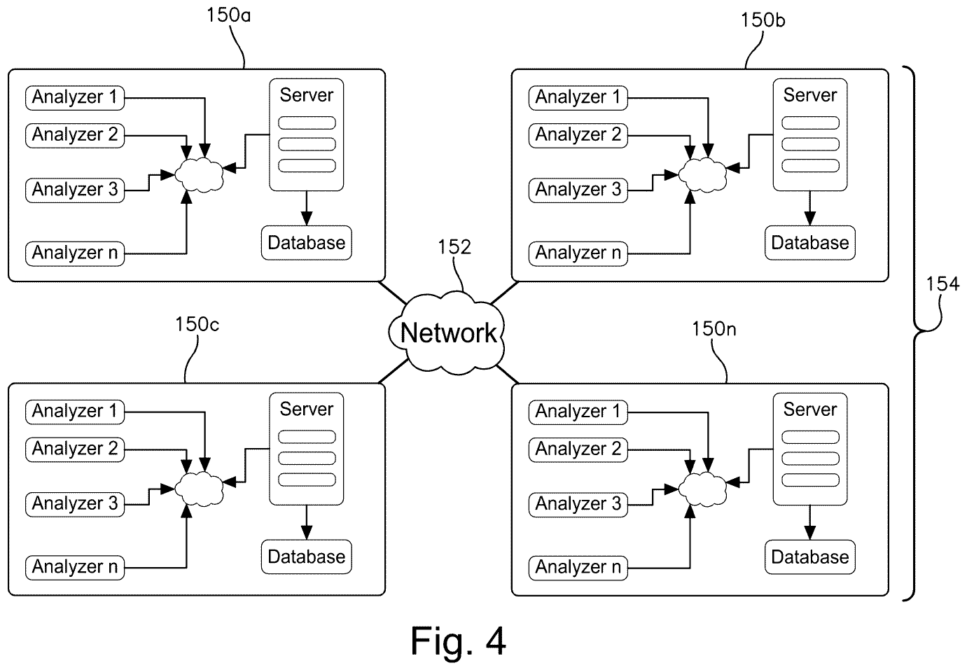

In one exemplary embodiment, as depicted in FIG. 4, a plurality of clinical diagnostics systems 150a, 150b, 150c, 150n, such as those depicted in FIG. 1, are in communication via a network, such as the Internet or other WAN. This collection of separate systems comprises a peer group of systems 152, wherein each system 150a, 150b, 150c, 150n represents a laboratory having one or more clinical diagnostic analyzers, and wherein each of the laboratories conducts testing of patient specimens and quality control materials. In some embodiments, each member 150a, 150b, 150c, 150n of the peer group 152 is a laboratory at a location geographically dispersed from the other peer group member laboratories, with each laboratory having similar types of clinical diagnostic analyzers, running similar types of tests and using quality control materials and/or patient specimens or analytes similar to those used by other peer members of the peer group.

Looking back to FIG. 1, server 112 preferably includes a processor 118, memory 120, and logic and control circuitry 122, all in communication with each other. Server 112 may be any server, server system, computer, or computer system known in the art, preferably configured to communicate instructions and data between the server 112 and the network, and/or to any device connected to the network, and to store and retrieve data and information to and from the database 114. Processor 118 may be any microprocessor, controller, or plurality of such devices known in the art. Processor 118 preferably runs a server operating system such as a Linux based, Windows based, or other server operating system known in the art. Preferably, the processor 118 is configured to control the operation of the server 112 in conjunction with the operating system, allowing the server to communicate with the database 114 and the network 116 and/or with devices connected to the network, such as the clinical diagnostic analyzers 110a, 110b, 110c, 110n. In some embodiments the server may control the operation of the clinical diagnostic analyzers, for example allowing operation of the analyzers during specific time periods, collecting data from the analyzers for storage in the database 114, transferring data to the analyzers for viewing and/or analysis, collecting test data from the analyzers, and providing data, instructions or prompts to the analyzers either individually or in groups.

Memory 120 may be volatile or non-volatile memory and is used to store data and information associated with the operation of the server as well as data for transmission to and from the server. For example, the memory stores the server operating system for execution by the processor 118 and may also store data associated with the clinical diagnostic analyzers 110a, 110b, 110c, 110n in communication with the server 112 over the network 116. In some embodiments the memory 120 on the server may be used as a supplement to, or in place of, the database 114.

The database 114 is preferably used to store control information relating to the operation of the server 112 and the operation and control of the clinical diagnostic analyzers 110a, 110b, 110c, 110n, and may also be used to store data relating to the processing of samples by the clinical diagnostic analyzers. For example, the database may contain instructions or programming for execution by a processor on a clinical diagnostic analyzer, or for execution on the server, or may store data related to the number of samples processed, the frequency of testing, the results of analysis performed on the analyzer, as well as data relating to the samples themselves, such as tracking information, lot numbers, sample size, sample weight, percentage of sample remaining, and the like. Preferably, the database 114 includes non-volatile storage such as hard drives, solid state memory, and combinations thereof.

Logic and control circuitry 122 provides interface circuitry to allow the processor and memory to communicate, and to provide other operational functionality to the server, such as facilitating data communications to and from the network 116.

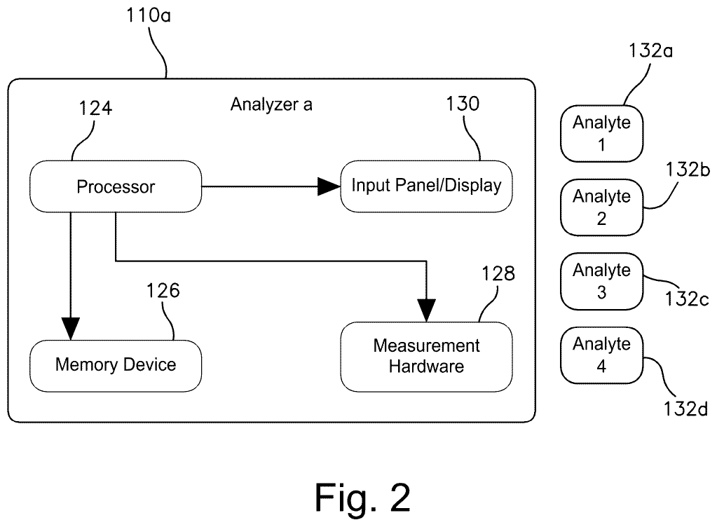

Turning to FIG. 2, a detailed view of a single clinical diagnostic analyzer 110a of the system of FIG. 1 is depicted. Clinical diagnostic analyzer 110a preferably comprises a processor 124, a memory device 126, measurement hardware 128, and an input panel/display 130.

The processor 124 may be any controller, microcontroller, or microprocessor as known in the art, and is in communication with memory device 126 which stores instructions for execution by the processor to control and communicate with the measurement hardware 128 and the input panel/display 130 to cause the clinical diagnostic analyzer to perform desired steps, such as sampling as commanding the measurement hardware to load test specimens or to perform a test on a loaded sample, or instructing or prompting an user to perform specific operations such as replacing a test sample, beginning a test, or viewing collected data. The processor 124 may also execute instructions to receive data from the measurement hardware 128 and to perform one or more analyses on the received data, and to display test results or other information on the input panel/display panel 130.

Measurement hardware 128 preferably includes a sample receptacle configured to receive one or more samples or specimens into the analyzer for testing. Preferably, the measurement hardware is configured to receive samples stored within vials, and most preferably is configured to receive a plurality of vials and to extract analytes, from any desired vial for and analysis. In further embodiments, the measurement hardware 128 may include external turntables, loaders, or other mechanisms to facilitate the loading and unloading of samples to allow samples to be loaded under command of the analyzer.

As depicted in FIG. 2, the measurement hardware is configured to be used with analyte samples 132a, 132b, 132c, 132d, which may be QC materials, patient test specimens or other specimens or analytes as is known in the art. In one embodiment, the material samples are contained in vials which are loaded or inserted into the clinical diagnostic analyzer 110a by a user. The samples may be loaded individually, or in groups, e.g., in a tray that is loaded into the analyzer. In alternative embodiments, the samples may be loaded using an automated loading mechanism, such as a turntable or other mechanism, upon command from the analyzer 110a. Material samples in the form of QC materials are typically provided in lots, with a unique lot number assigned to a lot of samples that are essentially identical as coming from the exact same batch source of material. The analyzer 110a preferably allows information relating to the QC materials to be entered by an user, including statistical information such as a mean or standard deviation for the lot of material. In other embodiments, the information may be obtained over a network or from a server using, for example a QR code on the sample vial or container to uniquely identify the sample or lot.

Input panel/display 130 is in communication with the processor and is operable to present controls to facilitate operation of the analyzer, as well as to present prompts and instructions to an user, and to receive input commands and/or data from the user. The input panel/display 130 is preferably a touch screen having capabilities of displaying text and graphics as well as icons, push buttons, keyboards, and the like to both present data to a user and to receive input from a user of the analyzer. Preferably, the input panel/display 130 includes an audible alert device such as a buzzer or beeper.

It should be understood that the clinical diagnostic analyzer 110a (as well as the analyzers 1 through n as depicted in FIG. 1) are not general purpose computers, but are specialized test equipment units wherein the measurement hardware 128 interacts with physical specimens being tested (i.e., analytes) to determine specific characteristics of the analyte. It should be further understood that the system and method of the present invention provide for an improvement to the technological field of clinical diagnostics and improve the operation of the technology by minimizing the likelihood of reporting erroneous patient results as compared to using individual instrument mean and SD values. Minimized numbers of erroneous patient results reduces retesting and saves time, labor, and material costs as compared to systems and methods known in the prior art.

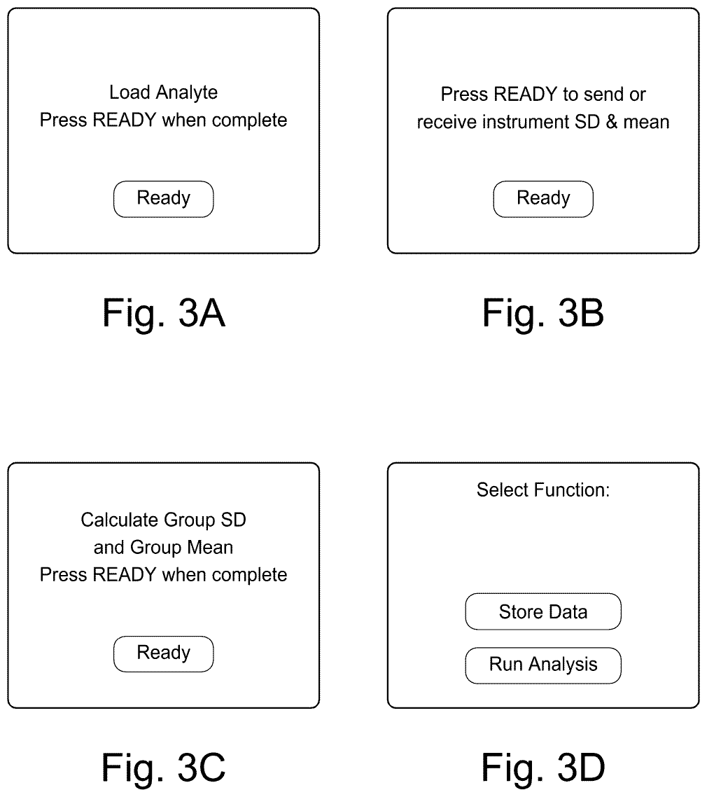

Looking to FIGS. 3A, 3B, 3C, and 3D, for example, the input panel/display may present prompts to a user to load an analyte to be tested and press a READY button once completed (FIG. 3A), to send or receive the SD (and/or mean) to or from another analyzer or server to allow calculation of a group SD and/or group mean as described in more detail below (FIG. 3B), to calculate a group SD and/or group mean (FIG. 3C) or to begin analysis of a loaded analyte or store data from an analysis (FIG. 3D). It should be understood that the clinical diagnostic analyzer 100a may have multiple programs and functions available, with a menu or selection prompt preferably presented to guide a user through the operation of the analyzer and the selection of desired functions and operations.

Clinical diagnostic analyzer 110a may be any type of analyzer known in the art, such as biochemistry analyzers, hematology analyzers, immune-based analyzers, or any other clinical diagnostic analyzer known in the art. Preferably, analyzer 110a is configured to test analytes such as patient specimens. Clinical diagnostic analyzer 110a may likewise be configured for use with various quality control materials, whether in liquid or lyophilized form, and may be configured for use in the immunoassay, serum chemistry, immunology, hematology, and other fields.

Looking to FIGS. 1 through 3 in combination, in typical use in designing and implementing QC ranges for multiple clinical diagnostic instruments testing the same analyte, the analyzer 110a prompts a user to load an analyte for testing as depicted in FIG. 3A. A user may also send or receive the mean and SD for the specific analyzer to another analyzer or server for calculation of a group mean and group SD as depicted in FIG. 3B, or may calculate the group SD and group mean as depicted in FIG. 3C. As is described in more detail herein, any one of the analyzers/instruments may receive the SD and mean from the other instruments and any one o the analyzers/instruments may perform the calculation to determine the group SD and group mean. It should be understood that the screen depictions in FIGS. 3A through 3D are exemplary in nature, and that other screens and/or transfer of data between the instruments may likewise be implemented in accordance with the present invention. As depicted in FIG. 3D, with the group SD and group mean loaded into the individual analyzer the user may run the analysis on the analyte. Upon completion of the test, the analyzer may prompt the user to store or review the data.

It should be understood that the operation of the analyzer 100a may be performed locally, at the analyzer, or that the operation may be coordinated thorough the server 112 when the analyzer is operated in a system 100 as depicted in FIG. 1 It should be further understood that any data may be stored locally on the analyzer 110a, on the server 112 or database 114, and that the data may be made available throughout the system 100 and over the network 115 so that remote servers and analyzers may likewise access the stored data. Similarly, analyses may be run on the analyzer itself, on the server, or may be distributed among multiple analyzers and/or servers.

It should also be understood that data collected and/or stored on any of the individual clinical diagnostic analyzers in any of the systems may be shared and communicated to other clinical diagnostic analyzers in that same system or laboratory, may be shared and communicated with the server and database within that system, and may be shared and communicated to other systems, and to the clinical diagnostic analyzers and servers and databases within those other systems.

Looking to FIG. 4, in one exemplary embodiment, a plurality of clinical diagnostics systems 150a, 150b, 150c, 150n, each of which are similar to that depicted in FIG. 1, are in communication via a network 152, such as the Internet or other WAN. This collection of separate systems comprises a peer group 154 of systems, wherein each system 150a, 150b, 150c, 150n represents a laboratory having one or more clinical diagnostic analyzers, and wherein each of laboratories conducts testing of patient specimens and quality control materials. Most preferably, each member 150a, 150b, 150c, 150n of the peer group 154 is a laboratory at a location geographically dispersed from the other peer group member laboratories, with each laboratory having similar types of clinical diagnostic analyzers, running similar types of tests and using quality control materials similar to those used by other members of the peer group.

In embodiments of the invention described herein, the analyses performed on multiple analyzers and the data collected by members of the peer group may be analyzed in combination to provide an output or result based on data collected across multiple analyzers, and based on data collected by other members of the peer group.

With the configuration of clinical diagnostic analyzers, systems employing clinical diagnostic analyzers, and peer groups of clinical diagnostic analyzers set forth, systems and methods for conducting virtual crossover studies in accordance with the present invention will now be described.

For a group of clinical diagnostic analyzers, each conducting an analysis on a single analyte, a group mean and group SD may be calculated based on the mean and SD of each individual analyzer as follows:

For n number of instruments/analyzers (e.g., analyzers 1 through n in FIG. 1), the group mean (μ) may be calculated as:

$$Group Mean = μ = {\sum }_{i = 1}^{n} \frac{1}{n} * {inst\_mean}_{i}$$

and the group standard deviation (SD) may be calculated as:

$$Group S D = \sqrt{{\sum }_{i = 1}^{n} ( \frac{1}{n} * {inst\_SD}_{i}^{2} ) + {\sum }_{i = 1}^{n} [ \frac{1}{n} * {( {inst\_mean}_{i} - μ )}^{2} ]}$$

Where:
inst_meanᵢ is the mean of an individual instrument i (for each of the instruments 1 through n), and inst_mean (without subscript) is a vector of all of the i individual instrument means; and
inst_SDᵢ is the standard deviation (SD) of the individual instrument i (for each of the instruments 1 through n) and inst_SD (without subscript) is a vector of all of the i individual instrument standard deviations.

The probability of false rejection for an individual instrument i and a given QC rule (QCrule) specified with a QC target and QC range may be calculated from the QC target, QC range, inst_meanᵢ, and inst_SDᵢ.

QCrulePfr(QC target, QC range, inst_meanᵢ, inst_SDᵢ) computes the false rejection rate for instrument i with a QC rule using the specified QC target and QC range;

For n number of instruments/analyzers:

$$Pfrgrp = {\sum }_{i = 1}^{n} \frac{1}{n} * QCrulePfr ( GroupMean , sr * GroupSD , {inst\_mean}_{i} , {inst\_SD}_{i} )$$

Pfrgrp is the average false rejection rate for a group of n instruments using a QC rule with QC target=GroupMean and QC range=sr*GroupSD
Where:
sr is a factor to be determined to give a desired false rejection rate (Pfr).

The value of sr is determined so that Pfrgrp=Pfr.

The value of sr may be calculated using any number of well-known algorithms, such as the Bisection Method.

Using the above equations and calculations in a manner as will be described below, the Group Mean and Group SD values may be provided to each of the individual clinical diagnostic analyzers in performing the testing/analysis on the analyte as described above with respect to FIGS. 3A through 3D. As also described above, using the group mean and group SD values (as opposed to each separate instrument using its own mean and SD values) minimizes the likelihood of reporting erroneous patient results as compared to using individual instrument mean and SD values. Minimized numbers of erroneous patient results reduces retesting and saves time, labor, and material costs as compared to systems and methods known in the prior art.

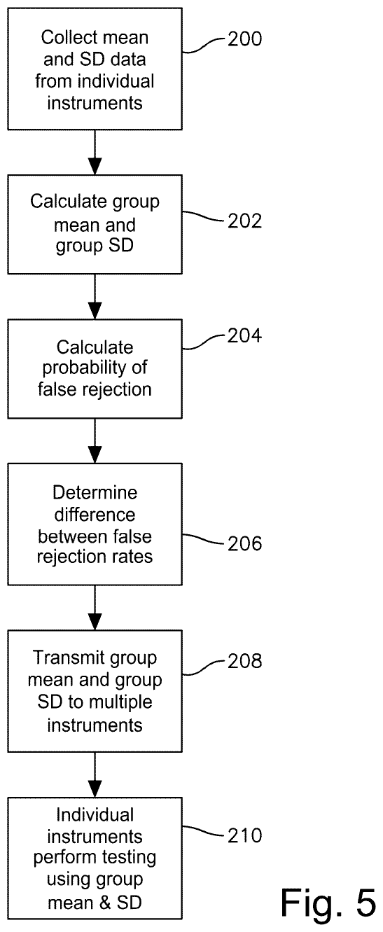

With the initial parameters and equations for calculating group mean and group SD values for testing an analyte on multiple instruments set forth, the steps for implementing the group mean and group SD on a group of instruments in accordance with an exemplary embodiment of the present invention are depicted in the flow diagram of FIG. 5.

Looking first to FIG. 5, a method for designing and implementing QC ranges for multiple clinical diagnostic instruments testing the same analyte begins at block 200. At block 200, the mean and SD from each individual instrument in the group of instruments being used to test the analyte are collected. As described above, it should be understood that the mean and SD data from each of the individual instruments may be collected at any one of the individual instruments or may be collected at a server, with further calculations on the collected data likewise performed at the individual instrument or server where the data is collected. Similarly, calculated or determined data may be sent or transmitted to the other instruments for use in testing the analyte as described herein.

At block 202, a group mean and a group SD value are calculated. As described above, the group mean and group SD values are calculated from the means and SDs of the individual instruments as:

$$Group Mean = μ = {\sum }_{i = 1}^{n} \frac{1}{n} * {inst\_mean}_{i} ; and \\ Group SD = \sqrt{{\sum }_{i = 1}^{n} ( \frac{1}{n} * {inst\_SD}_{i}^{2} ) + {\sum }_{i = 1}^{n} [ \frac{1}{n} * {( {inst\_mean}_{i} - μ )}^{2} ]}$$

At block 204 for a given QC rule and desired false rejection rate (Pfr) the probability of false rejection for that QC rule and false rejection rate may be calculated form the QC targe, QC range, and instrument mean and instrument SD vectors as described above.

At block 206 a difference between the Pfr of the instruments and the group of instruments may be calculated as described above.

With the desired group mean and group SD determined, at block 208 the group mean and group SD are sent to each of the individual instruments in the group for use by each instrument in testing the analyte, such that every instrument in the group testing the same analyte use the same group SD and group mean values.

At block 210, as depicted in FIG. 3D, each individual instrument in the group performs testing on the analyte loaded in the respective instrument, and the results may be stored or sent to other instruments or the server for display, generating alerts, further storage, or presentation to a user. It should be understood that each individual instrument in the group may perform its testing of the analyte at different times, with the data aggregated and stored as it becomes available—i.e., the testing at each individual instrument need not occur simultaneously.

Thus, as described herein, the design and implementation of testing of a single analyte on multiple individual clinical diagnostic analyzers or instruments is performed using a group mean and group SD rather than each instrument using the mean and SD for that individual instrument.

The system and method as described herein thus provides an improvement to the clinical diagnostic and analyte testing technology and minimizes the likelihood of reporting erroneous patient results as compared to using individual instrument mean and SD values. Minimized numbers of erroneous patient results reduces retesting and saves time, labor, and material costs as compared to systems and methods known in the prior art.

As can be seen, the systems and methods of the present invention provide an improvement over the generally accepted methods of testing an analyte among several individual instruments with the testing data merely aggregated afterward.

While the present invention has been described and illustrated hereinabove with reference to various exemplary embodiments, it should be understood that various modifications could be made to these embodiments without departing from the scope of the invention. Therefore, the invention is not to be limited to the exemplary embodiments described and illustrated hereinabove, except insofar as such limitations are included in the following claims.

What is claimed and desired to be secured by Letters Patent is as follows:

1. A clinical diagnostic analyzer for conducting virtual crossover studies, comprising:
a processor;
measurement hardware in communication with the processor and configured to measure properties of an analyte;
a memory device having stored thereon executable instructions that, when executed by the processor, cause the clinical diagnostic analyzer to perform operations comprising:
acquiring individual instrument mean and standard deviation data from each of a plurality of clinical diagnostic analyzers;
calculating a group mean and a group SD value from the acquired individual instrument mean and SD data;
transmitting the calculated group mean and group SD values to each of the plurality of clinical diagnostic analyzers;
loading the analyte on the clinical diagnostic analyzer;
testing the analyte on the clinical diagnostic analyzer using the group mean and group SD values;
collecting analyte test results from each of the plurality of clinical diagnostic analyzers; and
presenting analyte test results data on a display, generating an alert based on analyte test results data, storing analyte test results data, and combinations thereof.

2. The clinical diagnostic analyzer of claim 1, wherein the memory device includes instructions, that when executed, further cause the clinical diagnostic analyzer to perform operations comprising:
determining a difference between a false rejection rate of an individual instrument and a false rejection rate of the plurality of clinical diagnostic analyzers.

3. The clinical diagnostic analyzer of claim 1, wherein the memory device includes instructions, that when executed, further cause the clinical diagnostic analyzer to perform operations comprising:
determining a difference between a false rejection rate of an individual instrument and a false rejection rate of the plurality of clinical diagnostic analyzers.

4. The clinical diagnostic analyzer of claim 1, wherein the memory device includes instructions, that when executed, further cause the clinical diagnostic analyzer to perform operations comprising:
presenting a prompt on the input panel and display to a user to load a specimen into the measurement hardware; and
accepting an input from the user indicating that the specimen has been loaded.

5. The clinical diagnostic analyzer of claim 1, wherein the the memory device includes instructions, that when executed, further cause the clinical diagnostic analyzer to perform operations comprising:
alerting a user if the data if the analyte test results exceed a predetermined threshold.

6. The clinical diagnostic analyzer of claim 1, further comprising an input panel and display operable to present information and data from the processor to a user and to accept input and selections from a user.

7. A system for conducting testing of a single analyte on a group of clinical diagnostic analyzers, comprising:
a plurality of clinical diagnostic analyzers in communication with a server, wherein each of the plurality of clinical diagnostic analyzers comprises:
a processor;
measurement hardware in communication with the processor and configured to measure properties of an analyte;
a memory device having stored thereon executable instructions;
wherein the executable instructions stored on the memory device of at least one of the clinical diagnostic analyzers, when executed by the corresponding processor, cause the clinical diagnostic analyzer to perform operations comprising:
acquiring individual instrument mean and standard deviation (SD) data from each of the individual clinical diagnostic analyzers of the plurality of clinical diagnostic analyzers;
calculating a group mean and a group SD value from the individual instrument mean and SD data;
transmitting the calculated group mean and group SD values to each of the plurality of clinical diagnostic analyzers;
loading the analyte on each of the plurality of clinical diagnostic analyzers;
testing the analyte on each of the individual clinical diagnostic analyzers of the plurality of clinical diagnostic analyzers, wherein each of the individual clinical diagnostic analyzers uses the group mean and group SD values in testing;
collecting the analyte test results from each of the plurality of clinical diagnostic analyzers at a single clinical diagnostic analyzer; and
presenting analyte test results data on a display, generating an alert based on analyte test results data, storing analyte test results data, and combinations thereof.

8. The system of claim 7, wherein the memory device includes instructions, that when executed, further cause the clinical diagnostic analyzer to perform operations comprising:
calculating a probability of false rejection for a given QC rule and a desired false rejection rate.

9. The system of claim 7, wherein the memory device includes instructions, that when executed, further cause the clinical diagnostic analyzer to perform operations comprising:
determining a difference between a false rejection rate of an individual instrument and a false rejection rate of the plurality of clinical diagnostic analyzers.

10. The system of claim 7, wherein the memory device includes instructions, that when executed, further cause the clinical diagnostic analyzer to perform operations comprising:
presenting a prompt on the input panel and display to a user to load the analyte into the measurement hardware; and
accept an input from the user indicating that the specimen has been loaded.

11. The system of claim 7, wherein the memory device includes instructions, that when executed, further cause the clinical diagnostic analyzer to perform operations comprising:
alerting a user if the data if the analyte test results exceed a predetermined threshold.

12. The system of claim 7, further comprising an input panel and display operable to present information and data from the processor to a user and to accept input and selections from a user.

13. A method for conducting testing of a single analyte on a group of clinical diagnostic analyzers, comprising:
acquiring individual instrument mean and standard deviation (SD) data from each of the individual clinical diagnostic analyzers of the plurality of clinical diagnostic analyzers;
calculating a group mean and a group SD value from the individual instrument mean and SD data;
transmitting the calculated group mean and group SD values to each of the plurality of clinical diagnostic analyzers;
loading the analyte on each of the plurality of clinical diagnostic analyzers;
testing the analyte on each of the individual clinical diagnostic analyzers of the plurality of clinical diagnostic analyzers, wherein each of the individual clinical diagnostic analyzers uses the group mean and group SD values in testing;
collecting the analyte test results from each of the plurality of clinical diagnostic analyzers at a single clinical diagnostic analyzer; and
presenting analyte test results data on a display, generating an alert based on analyte test results data, storing analyte test results data, and combinations thereof.

14. The method of claim 13, further comprising:
calculating a probability of false rejection for a given QC rule and a desired false rejection rate.

15. The method of claim 13, further comprising:
determining a difference between a false rejection rate of an individual instrument and a false rejection rate of the plurality of clinical diagnostic analyzers.

16. The method of claim 13, further comprising:
presenting a prompt on an input panel and display of a clinical diagnostic analyzer to a user to load the analyte into the measurement hardware; and
accepting an input from the user indicating that the specimen has been loaded.

17. The method of claim 13, further comprising:
alerting a user if the data if the analyte test results exceed a predetermined threshold.
